(19) United States
(12) Patent Application Publication
(10) Pub. No.: US 2026/0215669 A1
(43) Pub. Date: Jul. 30, 2026

(54) AN ENDOSCOPE DEVICE AND IMAGING METHOD FOR INTRAOPERATIVE IMAGING OF THE COCHLEA AND IMAGING-GUIDED COCHLEAR IMPLANTATION

(71) Applicants: The General Hospital Corporation, Boston, MA (US); The Board and Trustees of the Leland Stanford Junior University, Stanford, CA (US)

(72) Inventors: Fang Hou, Stoneham, MA (US); Radhika Poduval, Somerville, MA (US); Alejandro E. Ortega, Boston, MA (US); Guillermo Tearney, Cambridge, MA (US); Konstantina Stankovic, Palo Alto, CA (US)

(21) Appl. No.: 19/150,877

(22) PCT Filed: Jan. 26, 2024
(86) PCT No.: PCT/US2024/013167

§ 371 Date: Jul. 24, 2025

Related U.S. Application Data
(60) Provisional application No. 63/481,625, filed on Jan. 26, 2023.

(51) Int. Cl. A61B 1/07; A61B 1/00; A61B 1/06; A61B 34/30; A61B 90/00

(52) U.S. Cl. CPC A61B 1/07; A61B 1/00126; A61B 1/0627; A61B 34/30; A61B 90/37; A61B 2034/301; A61B 2090/3735

(57) ABSTRACT

An endoscope system, including: an optical imaging probe having a proximal end and a distal end, including a fiber connector, an optical probe position module, an optical fiber, and an optical probe head, configured such that the optical probe head delivers a focused light beam, wherein the optical probe position module controls an axial position of the optical probe head; and a sheath having a proximal end and a distal end, comprising a flexible sheath, a tube adaptor, and a stylet tube, wherein the flexible sheath, tube adaptor, and stylet tube are configured to form a continuous channel and surround at least part of the optical fiber.

### CROSS-REFERENCE TO RELATED APPLICATIONS

The present application claims the benefit of priority to U.S. Provisional Application No. 63/481,625, filed Jan. 26, 2023, the contents of each of which are incorporated herein by reference in their entirety.

### STATEMENT REGARDING FEDERALLY SPONSORED RESEARCH OR DEVELOPMENT

This invention was made with government support under W81XWH-20-1-0855 awarded by the US Army Medical Research Acquisition Activity. The government has certain rights in the invention.

### BACKGROUND

There is a large sensorineural hearing loss (SNHL) population. As of today, the most successful treatment method for SNHL is cochlear implantation. Cochlear implantation procedure has the risks of damaging the dedicated structure of the cochlea and may damage the patient's residual hearing. The electrode array is the component of a cochlear implant that is inserted into the cochlea. The insertion of the electrode array is guided mostly based on the experience of the operating surgeon with limited or no intraoperative guidance. There are also currently no in vivo imaging methods available to make a diagnosis of the pathological change in the cochlea, so the outcomes of cochlear implantation are difficult or impossible to correlate to the patients' cochlea pathology, and the prognosis is unclear.

Manufacturers are improving the design and introducing different approaches, including different assistant devices to help with insertion to mitigate the risk of damage to the cochlea tissue. One such approach is the “advance off-stylet” technique, which is a cochlear implant electrode array insertion technique in which the electrode arrays are designed to have a channel in the center of the body and a solid metal stylet is pre-inserted into the channel to increase the assembly's rigidness for easier insertion and mitigate the chance of insertion trauma. Nevertheless, many challenges remain.

### SUMMARY

Disclosed herein are methods and systems for intraoperative imaging of the cochlea and imaging-guided cochlear implant insertion, including an endoscope system. In various embodiments, the methods and systems may include one or more of the following.

An endoscope system is disclosed. The endoscope device includes an optical imaging probe and a sheath. The optical imaging probe has a proximal end and a distal end. Similarly, the sheath has a proximal end and a distal end. In some embodiments, the optical imaging probe includes a fiber connector, an optical probe position module, an optical fiber, and an optical probe head. The optical imaging probe is configured such that the optical probe head can deliver a focused light beam at a sample. The optical probe position module controls the position of the optical imaging probe. The sheath comprises a flexible sheath, a tube adaptor, and a stylet tube. The flexible sheath, tube adaptor, and stylet tube are configured to form a continuous channel and surround at least part of the optical fiber.

A method of imaging a human cochlea in vivo is also disclosed. The method includes providing an endoscope device. The endoscope includes an optical imaging probe and a sheath. The endoscope is configured such that an optical probe head at the distal end of the optical imaging probe can extend beyond the distal end of the sheath. The method further includes disposing the endoscope device into the stylet channel of an electrode array and advancing the endoscope within the electrode array. The method further includes inserting the electrode array-endoscope device into the cochlea. Inserting may further include emitting light into the proximal end of the optical image probe such that the distal end of the optical probe head emits light toward the interior of the cochlea and focus on the tissues of the cochlea. Inserting the endoscope device may also include rotating the optical imaging probe to focus light on different locations of the tissue of the cochlea. Inserting the endoscope may also include causing the optical imaging probe to receive reflected light from the cochlea and transmit the reflected light toward the proximal end of the optical imaging probe. Inserting may further include generating a two-dimensional image of a portion of the cochlea surrounding the optical imaging probe based on the reflected light received from the optical imaging probe. Essentially, inserting may include imaging the interior of the cochlea while inserting. The method may further include causing the two-dimensional image of the portion of the cochlea surrounding the optical probe to be presented. The two-dimensional image may be indicative of a distance between an outer surface of the optical imaging probe and a wall of the cochlea. The two-dimensional image may provide cross-sectional structural and functional information of the tissue, including sensory cells inside the cochlea. The two-dimensional image may provide diagnostic information of SNHL and be used to corelate to the outcomes of cochlea implantation.

A method of intraoperative imaging-guided cochlear implantation is also disclosed. The method comprises providing an endoscope device. The endoscope includes an optical imaging probe and a sheath. The endoscope is configured such that an optical probe head at the distal end of the optical imaging probe can extend beyond the distal end of the sheath. The method further includes disposing the endoscope device into the stylet channel of an electrode array and advancing the endoscope within the electrode array. The method further includes inserting the electrode array-endoscope device into the cochlea. Inserting may further include emitting light into the proximal end of the optical image probe such that the distal end of the optical probe head emits light toward the interior of the cochlea and focus on the tissues of the cochlea. Inserting the endoscope device may also include rotating the optical imaging probe to focus light on different locations of the tissue of the cochlea. Inserting the endoscope may also include causing the optical imaging probe to receive reflected light from the cochlea and transmit the reflected light toward the proximal end of the optical imaging probe. Inserting may further include generating a two-dimensional image of a portion of the cochlea surrounding the optical imaging probe based on the reflected light received from the optical imaging probe. Essentially, inserting may include imaging the interior of the cochlea while inserting. Inserting the electrode array-endoscope device may also include identifying an insertion depth based on the generated two-dimensional images of the cochlea. The method also includes advancing the electrode array off of the stylet tube, during which the optical imaging probe continuing rotating and imaging to monitor the position of the electrode array and the cochlea.

### BRIEF DESCRIPTION OF THE DRAWINGS

FIG. 1 is a schematic of an endoscope device, in accordance with one or more embodiments of the disclosure. The endoscope device comprises an optical imaging probe (gray) and a sheath (black).

FIG. 2 is a schematic of an optical imaging probe, in accordance with one or more embodiments of the disclosure.

FIG. 3 is a schematic of assembly of a sheath, in accordance with one or more embodiments of the disclosure.

FIG. 4 is a schematic of an assembly of an endoscope device, in accordance with one or more embodiments of the disclosure.

FIG. 5 shows a schematic of electrode array-endoscope device assembly in accordance with one or more embodiments of the disclosure.

FIG. 6 shows a schematic of an optical probe head with multiple optical surfaces that generates multiple light propagation modes, in accordance with one or more embodiments of the disclosure.

FIG. 7 shows a schematic of an optical probe head directing light from a second waveguide comprising two segments of multimode fibers with different core diameters, in accordance with one or more embodiments of the disclosure.

FIG. 8 is a schematic of a sheath, in accordance with one or more embodiments of the disclosure.

FIG. 9 shows an embodiment of a tube adaptor (top) and an assembly of a flexible sheath, tube adaptor, and stylet (bottom), in accordance with one or more embodiments of the disclosure.

FIG. 10 is a schematic of an optical fiber interacting with an optical probe head, in accordance with one or more embodiments of the disclosure.

FIG. 11 is a schematic of a sheath with a stylet tube exposed from a tube adaptor, in accordance with one or more embodiments of the disclosure.

FIG. 12 shows a schematic (top) and cross-sectional view (bottom) of an optical position adjustment module, in accordance with one or more embodiments of the disclosure.

FIG. 13 shows a schematic of an endoscope device configured with a rotary joint and OCT console in accordance with one or more embodiments of the disclosure.

FIG. 14 shows a flow chart of a method of imaging the interior of the cochlea in accordance with one or more embodiments of the disclosure.

FIG. 15 shows a flow chart of a method of intraoperative imaging-guided cochlear implantation.

### DETAILED DESCRIPTION

In accordance with some embodiments of the disclosed subject matter, mechanisms (which can include, for example, systems and methods) for intraoperative imaging of the cochlea and imaging-guided cochlear implantation are provided.

In some embodiments, an endoscope device including an optical imaging probe and a sheath used to image the interior of a cochlea is described. In some embodiments, an optical imaging probe is disposed into a sheath prior to being inserted into a cochlea. In some embodiments, a sheath protects an optical imaging probe from damage. In some embodiments, an optical imaging probe can rotate within the sheath without contacting the inside of the sheath. In some embodiments, the optical imaging probe can move axially within a sheath and be advanced beyond the distal end of the sheath. In some embodiments, the endoscope is used with an electrode array of a cochlear implant. The endoscope is inserted into the channel of the electrode array prior to imaging. The stylet tube must be rigid to provide the mechanical support of the electrode array to perform the “advance off-stylet” insertion. Traditional stylets are made of metal to achieve this rigidness, which is not transparent to OCT light. Therefore, the optical imaging probe must be able to extend beyond the stylet tube in order to perform OCT imaging. The endoscope device is able to function as a traditional stylet tube and simultaneously perform OCT imaging. This helps guide insertion and mitigate risk of damaging the cochlea during insertion.

In some embodiments, a method of using an endoscope device to obtain cross-sectional OCT (optical coherence tomography) images of the interior of the cochlea in humans is described. OCT images of the cochlea can provide information of the sensory epithelium and morphology of the interior of the cochlea.

In some embodiments, a method to implant the electrode arrays of cochlear implant systems with the assistance of the endoscope device under real-time imaging guidance provided by the optical imaging probe of the endoscope device is described. In some embodiments, the stylet tube can serve as a traditional electrode array stylet that provides support of electrode arrays to assist with the insertion of the electrode array to the straight portion of the scala tympani without bending the electrode array or damaging the tissue inside the cochlea. The endoscope device will be inserted into the electrode arrays before the insertion of the whole assembly. During the insertion of the endoscope device-electrode array assembly, the optical imaging probe will be extended out from the rigid stylet tube within a safe range so that the optical probe head will not touch the electrode arrays. The optical imaging probe will rotate inside the sheath, driven by the driveshaft assembly, and obtain OCT images through the electrode arrays to get real-time feedback on the distance of the edge of the electrode arrays to the tissues in the scala tympani. Thus, the insertion procedure can be guided by real-time OCT imaging.

In some embodiments, the systems and methods described herein provide cellular-level visualization of the interior of the cochlea in humans. This can potentially provide diagnostic information for sensorineural hearing loss based on disease etiology and progression. The information provided by the OCT images can be used to correlate to the outcomes of cochlea implantation. This can also provide prognosis for cochlear implantation treatment. In some embodiments, the systems and methods described herein can aid in customizing future cochlear implant devices.

In some embodiments, the systems and methods described herein can help to minimize the risk of damaging the dedicated structure of the cochlea during the process of cochlear implant insertion, and thus avoid causing damage to a subject's residual hearing. In some embodiments, the systems and methods can improve the accuracy of where cochlear implants are inserted.

An endoscope system refers to a system or device that is used to image inside a body cavity and organ. The terms “endoscope system” and “endoscope device” are used interchangeably herein. In some embodiments, the endoscope system can be used to image the interior of a cochlea in a human.

OCT imaging refers to optical coherence tomography, which is a procedure for collecting high resolution cross-sectional images of a sample. Micro-OCT (μOCT) refers to a procedure for OCT imaging that increases the resolution of images of a sample. The terms “OCT” and “micro-OCT” are used interchangeably herein. OCT imaging is suitable for collecting images of the interior of a cochlea of a human. Micro-OCT imaging has a high lateral resolution (e.g., <5 μm) and maintains a long imaging range (e.g., >300 μm).

A “sample” refers to an object or tissue that is being imaged by an endoscope device. For instance, a sample may refer to the scala tympani of a human cochlea. A sample may be collected from a subject, such as a human. In some embodiments, a subject is a human who has been diagnosed with sensorineural hearing loss. In some embodiments, a subject is a human seeking to undergo a cochlear implant insertion procedure. In some embodiments, a subject is a human with symptoms of sensorineural hearing loss.

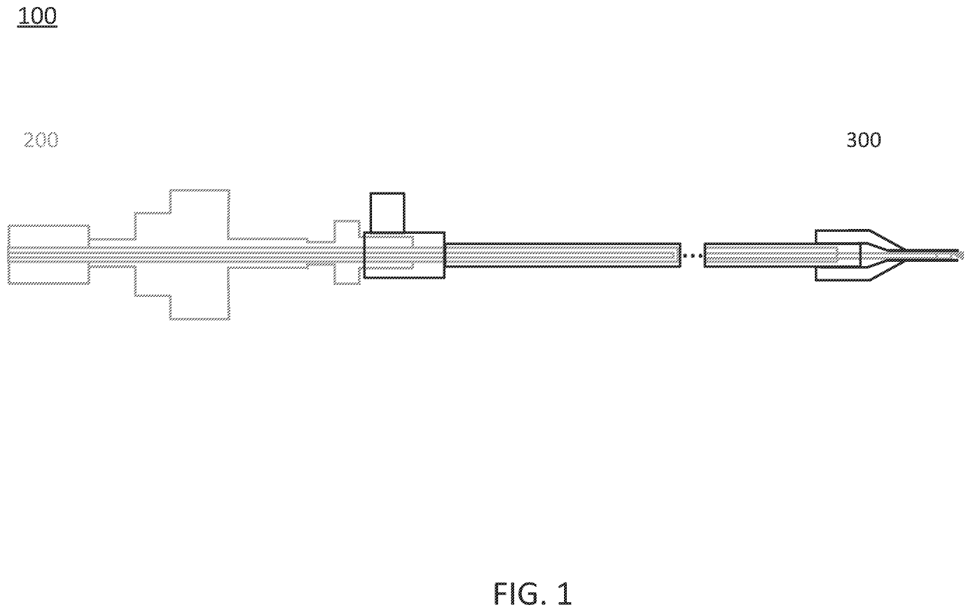

FIG. 1 shows an example 100 of an endoscope system for optical coherence tomography imaging of the cochlea in accordance with some embodiments of the disclosed subject matter. As shown in FIG. 1, endoscope device 100 comprises an optical imaging probe 200 and a sheath 300.

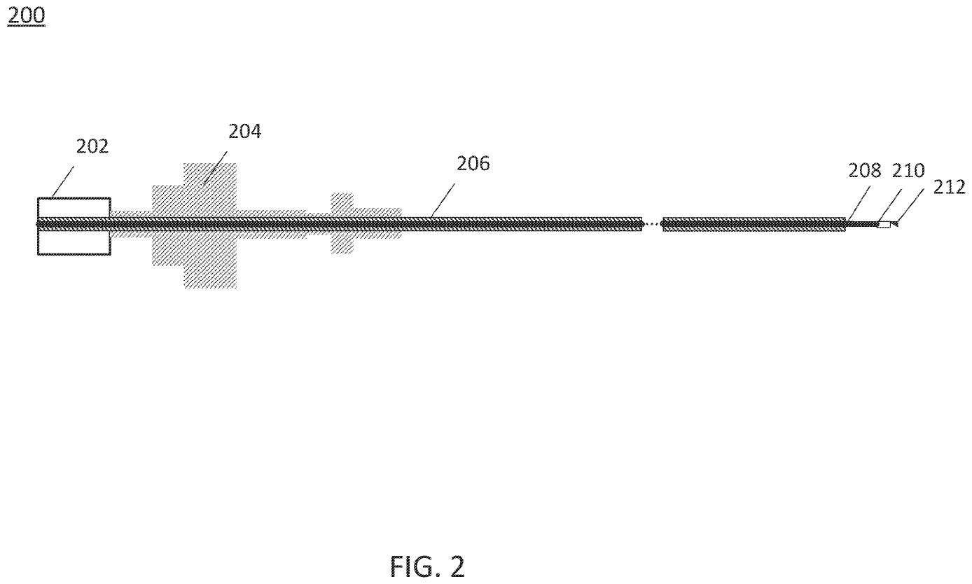

FIG. 2 shows an example 200 of an optical imaging probe in accordance with some embodiments of the disclosed subject matter. Optical imaging probe 200 can perform optical coherence tomography (OCT), an imaging method that allows the production of high-resolution cross-sectional imaging. It is particularly suitable for imaging the interior of a cochlea. Multiple optical set ups are suitable for this method of imaging. Some suitable optical systems and methods are described in US2019/0029570 A1, which is hereby incorporated by reference herein in its entirety.

As shown in FIG. 2, optical imaging probe 200 comprises a fiber connector 202 that delivers imaging light into an optical fiber and eventually delivers a focused light beam from the distal end of an optical imaging probe 200 on a sample (e.g., tissue of the interior of a cochlea) for imaging. An optical fiber can refer to a single waveguide, or a combination of multiple waveguides. An optical probe position adjustment module 204 connects the fiber connector 202 at the proximal end of the optical probe position adjustment module and the sheath 300 via the lockable sheath connector 302 at the distal end of the optical probe position adjustment module. The optical probe position adjustment module 204 houses a driveshaft assembly 206. The optical probe position adjustment module 204 holds sheath 300 when driveshaft assembly 206 is rotating. Optical probe position adjustment module 204 controls the axial position of the optical probe head 212. As used herein, the term axial position refers to a position along the axis parallel to the length of the endoscope device 100, such that the optical probe position adjustment module 204 controls the position of the optical probe head 212 along the length of the probe and in particular helps extend or retract the optical probe head 212 at the end of the probe.

A segment of driveshaft assembly 206 at its proximal end is housed within optical probe position adjustment module 204. Driveshaft assembly 206 connects to fiber connector 202 at the proximal end of driveshaft assembly 206. Driveshaft assembly 206 houses a first waveguide 208 within it. First waveguide 208 is housed inside fiber connector 202 and driveshaft assembly 206. A distal end of first waveguide 208 extends beyond the distal end of driveshaft assembly 206 for a fixed distance. In some embodiments, the fixed distance is equal to or less than (e.g., not longer than) the length of the channel of an electrode array endoscope device 100 may be used with. A second waveguide 210 can be spliced to first waveguide 208. In some embodiments, the combination of first waveguide 208 and second waveguide 210 is referred to as an optical fiber. An optical probe head 212 may be manufactured on the distal tip of second waveguide 210. Optical probe head 212 reflects and focuses the imaging light beam onto a sample (e.g., a portion of the scala tympani within the cochlea of a human subject). Optical probe head 212 can act as a focusing element in the optical imaging probe.

Optical imaging probe 200 is suitable for micro-optical coherence tomography. Optical imaging probe 200 can be configured to receive source light and reflect a portion of the light into a proximal end of first waveguide 208. In some embodiments, first waveguide 208 and second waveguide 210 can be configured to transmit the source light from the proximal end of first waveguide 208 to the distal end of second waveguide 210, which is coupled to optical probe head 212 and arranged to project the source light from optical probe head 212. Optical probe head 212 can be configured to reflect the source light emitted from distal end of first waveguide 208 and second waveguide 210 onto a sample.

In some embodiments, first waveguide 208 and second waveguide 210 can be arranged to receive reflected light from a sample via a reflective surface. The reflected light can then be transmitted along first waveguide 208 and second waveguide 210 from distal end to proximal end. Light reflected from sample that is transmitted to the proximal end can then be transmitted to an OCT imaging console, which includes an interferometer and an optical detector. The optical detector includes a camera sensor, such as a CCD image sensor, a CMOS image sensor, based on spectrometer for spectral domain OCT and high speed photo-diodes (e.g., made of silicon, germanium, InGaAx, lead sulfide, or other materials including balanced photo-diodes) for swept-source OCT. An OCT imaging console can be configured to receive both reference source light and light reflected from a sample which allows discrimination between signals from different depths using any suitable coherence tomography techniques.

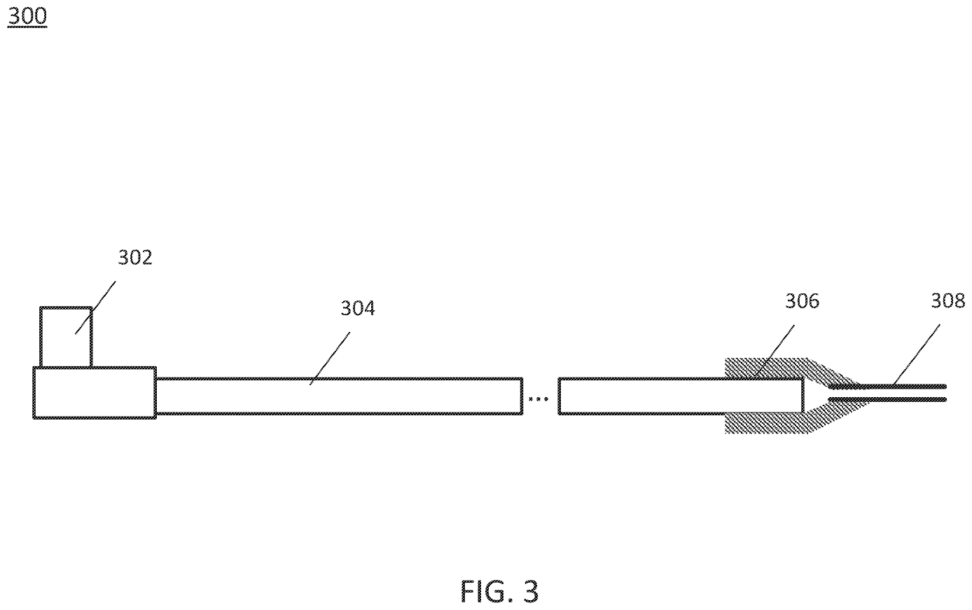

FIG. 3 shows an example of a sheath 300. Sheath 300 includes a lockable sheath connector 302 that connects the sheath to optical probe position adjustment module 204. Sheath 300 can also include a flexible sheath 304. Flexible sheath 304 can be made from materials that have a relatively small amount of friction and may include a Teflon-coated driveshaft (e.g., high density polyethylene, also referred to as HDPE). In some embodiments, sheath 300 can include a tube adaptor 306. At a proximal end of tube adaptor 306, tube adaptor 306 connects to flexible sheath 304. At a distal end of tube adaptor 306, tube adaptor 306 connects to a stylet tube 308. Tube adaptor 306 is made of materials that have high strength (e.g., stainless steel, ABS, PLA) so it can hold stylet tube 308 and resist any reasonable force applied while inserting stylet tube 308 (e.g., into a cochlea) without breaking. In some embodiments, stylet tube 308 can support an electrode array. Stylet tube 308 may be made of metal (e.g., stainless steel) and may be rigid. Stylet tube 308 may be at least 20 millimeters.

Flexible sheath 304, tube adaptor 306, and stylet tube 308 can form a continuous channel and surround at least part of optical imaging probe 200. Flexible sheath 304 can protect driveshaft assembly 206, while still allowing driveshaft assembly 206 to freely rotate. Stylet tube 308 can surround and protect elements such as the distal end of first waveguide 208, second waveguide 210, and optical probe head 212. Stylet tube 308 is particularly important to ensure that optical probe head 212 is not damaged when endoscope device 100 is inserted into a cochlea.

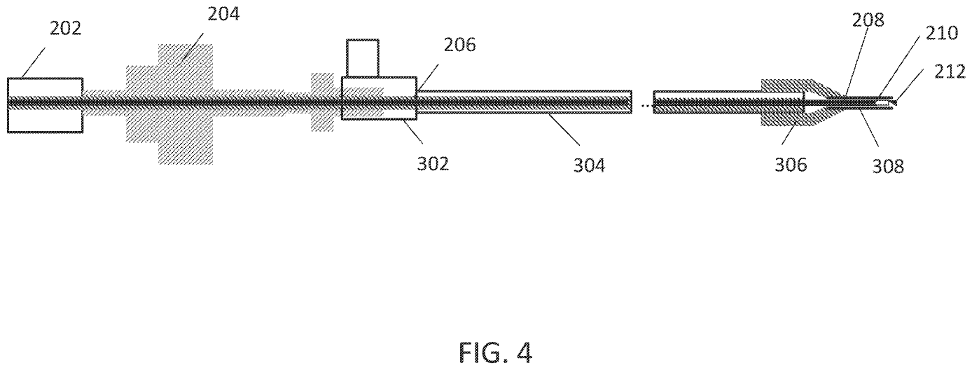

FIG. 4 shows a detailed schematic of endoscope device 100. A configuration of the components of optical imaging probe 200 and sheath 300 in accordance with one or more embodiments of the device is demonstrated. Lockable sheath connector 302 is attached to the distal end of optical probe position adjustment module 204. Flexible sheath 304 houses and protects driveshaft assembly 206 inside, and driveshaft assembly 206 can rotate within flexible sheath 304. The extended first waveguide 208 passes through tube adaptor 306 and stylet tube 308. The axial distance of the optical imaging probe 200 is controlled by optical probe position adjustment module 204. Optical probe adjustment module 204 controls the axial position of optical probe 200 by moving sheath 300 relative to optical probe 200 (e.g., optical imaging probe 200 remains still while sheath 300 moves).

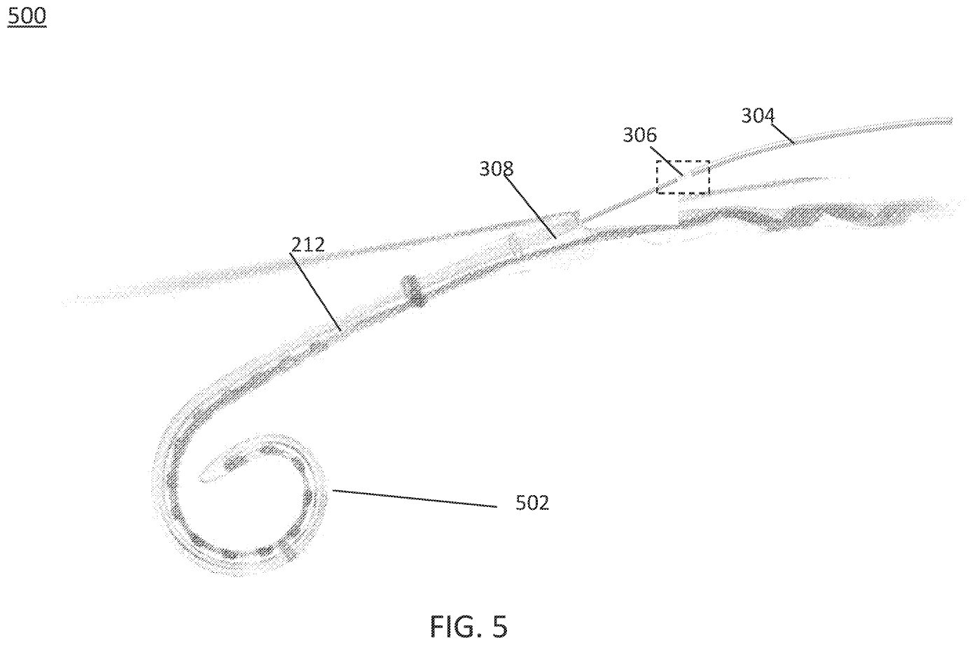

In some embodiments, endoscope device 100 will be used in combination with a cochlear implant electrode array. FIG. 5 shows an example of an electrode array-endoscope device 500 assembly in accordance with one or more embodiments of the disclosure. Before an imaging session, endoscope device 100 will be inserted into the channel of a cochlear implant electrode array 502. During this procedure, optical probe head 212 is secured and protected inside stylet tube 308 so it will not be damaged by the insertion. Optical imaging probe 200 will then be advanced in sheath 300. The tip of optical imaging probe 200, specifically optical probe head 212, will stretch out from stylet tube 308. Once optical probe head 212 reaches its ideal position, optical imaging probe 200 can be secured. Optical imaging probe 200 can then rotate inside the electrode array's center channel without touching the inner wall of the electrode array.

In some embodiments, stylet tube 300 is transparent to the OCT light. After endoscope device 100 is inserted into the ideal position in electrode array 502, optical imaging probe 200, specifically optical probe head 212 does not extend beyond stylet tube 308. Optical probe head 212 is rotated inside stylet tube 308. The axial position of optical probe head 212 is controlled by optical probe adjustment module 204. In some embodiments, optical probe adjustment module 204 slowly retracts optical probe head 212 during imaging so the OCT light performs a helical scan on the tissue inside the cochlea to obtain three-dimensional OCT images of the interior of the cochlea.

Various embodiments of optical imaging probe 200 can produce different imaging modalities. In some embodiments, first waveguide 208 can be a single-mode fiber. In some embodiments, second waveguide 210 can be a multimode fiber. In some embodiments, second waveguide 210 has a larger core diameter (e.g., larger than the core diameter of first waveguide 208) that allows the light beam emitted from first waveguide 208 to expand before reaching optical probe head 212. In some embodiments, second waveguide 210 can also modulate the optical beam modes so multiple foci can be generated after optical probe head 212 for the purpose of extending the depth of focus such that the depth of focus is longer than typical Rayleigh range of a focusing element.

Optical probe head 212 is not limited to the embodiments shown above and in various embodiments can include an optical assembly configured with miniature optical components, including but not limited to one or more of a multimode fiber, GRIN fiber, coreless fiber, ball lens, C-lens, and/or micro prisms. Optical probe head 212 can be made from 3D-printed components that focus and reflect the light beam to the tissue to enable production of OCT images.

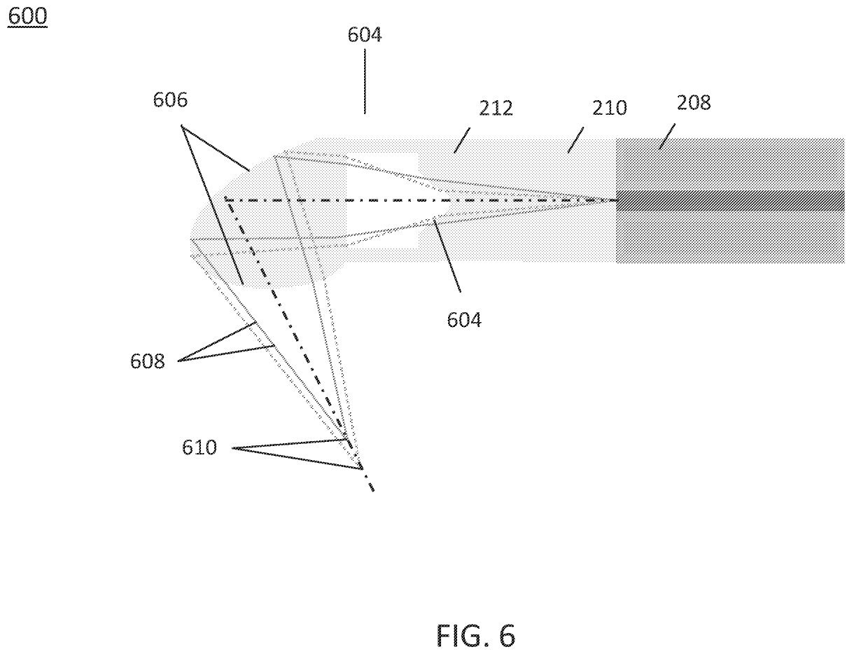

FIG. 6 shows one possible embodiment of optical probe head 212. Optical probe head 212 may be 3D printed optics 602. 3D printed optics 602 may be a monolithic structure that has multiple optical surfaces 604. 3D printed optics 602 may also have free form surfaces 606 (e.g., surfaces that can be digitally constructed to fit a specific probe and application and custom made, for example using 3D printing, and are not required to be based on mathematical functions such as polynomial, spherical, or elliptical functions) that can be customized to direct the light in various ways based on a desired output beam, including to generate multiple propagation modes. One multi-curvature surface could generate multiple light propagation modes 608 and the beam can be focused to multiple foci 610 within the tissue of the cochlea, thus generating an extended imaging depth of focus compared to traditional single optical elements that can only generate one focal point with a limited depth of focus that is generally defined by the Rayleigh range.

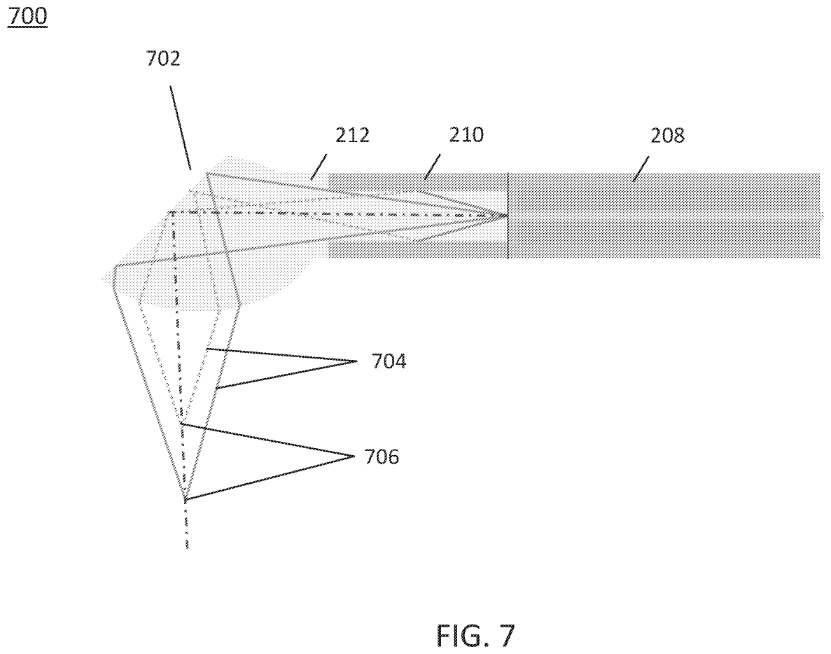

FIG. 7 shows another embodiment of optical probe head 212. In this embodiment, second waveguide 210 further includes two segments of multimode fibers with different core diameters. A first segment has a smaller core diameter that generates multiple internal reflection of the light beam. A second segment allows the beams to expand before reaching the focusing element. The optical probe head is a 3D printed monolithic structure 702, with two optical surfaces to reflect and focus multiple light beams 704 to the tissue. The light beams will be focused to multiple foci 706, thus the probe will have extended depth of focus compared to traditional single-lens optics.

In some embodiments, a 3D printed optical probe head has a smaller overall diameter than an optical fiber (e.g., <80 μm) so it will not touch the wall of the electrode array when the optical imaging probe 200 is rotating within stylet tube 308.

In some embodiments of sheath 300, the continuous channel has a wider channel at the proximal end and is reduced to a narrower diameter at the distal end. In some embodiments, the reduction in diameter happens within the tube adaptor. In some embodiments, the outer diameter of a distal end of the tube adaptor is smaller than an outer diameter of a proximal end of the tube adaptor.

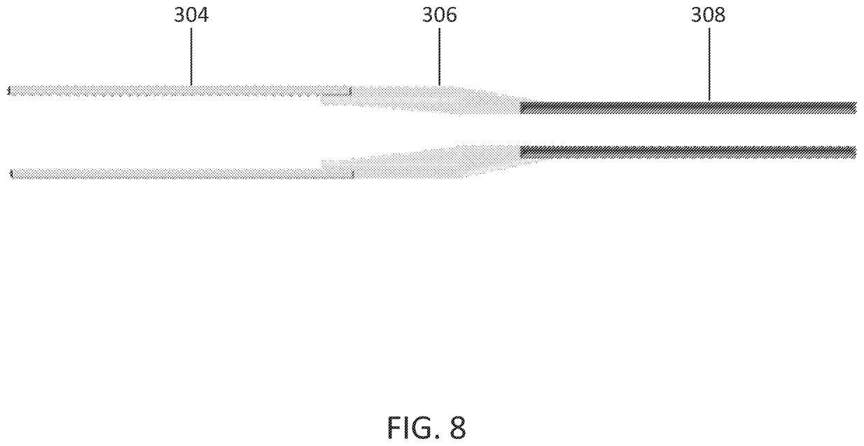

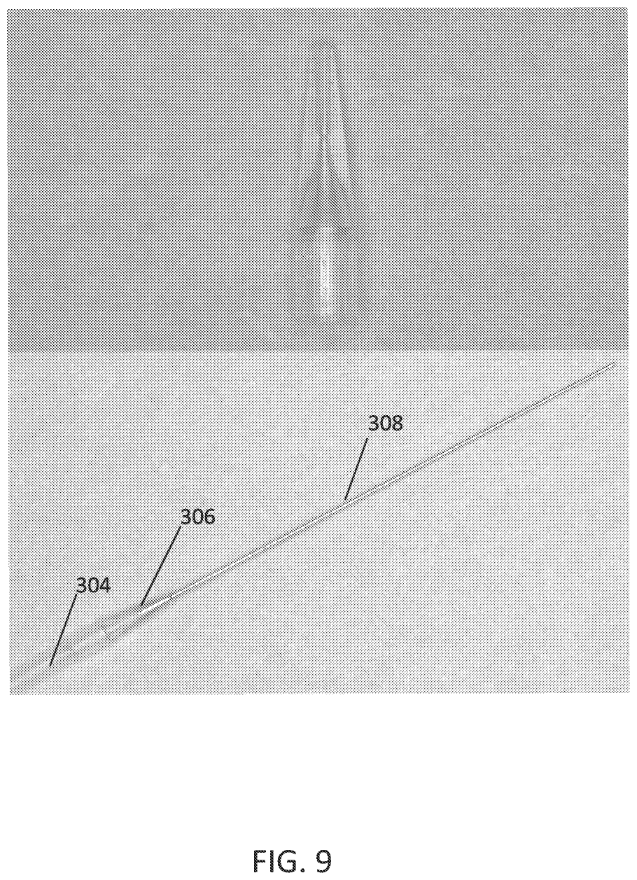

In one embodiment of sheath 300, tube adaptor 306 is a 3D printed part, having a hollow channel within its body. The proximal end of the tube adaptor 306 fits and can be assembled to the end of flexible sheath 304, and its distal end can be assembled to hold stylet tube 308. In the middle part of tube adaptor 306, the internal channel tapers from a larger diameter to a smaller diameter with a smooth transitional surface. FIG. 8 shows a schematic of the assembly flexible sheath 304, tube adaptor 306, and stylet tube 308. FIG. 9 shows photos of an embodiment of tube adaptor 306 (top panel) and the assembly of flexible sheath 304, tube adaptor 306, and stylet tube 308 (bottom panel). In some embodiments, has a clear inner lumen, which allows for smooth insertion of an optical fiber.

Endoscope system 100 has several parameters that are important for its application. The outer diameter of stylet tube 308 is equal to or smaller than 150 μm, which is a typical cavity size in current commercially available cochlear implant electrode arrays. This allows endoscope system 100 to work in an assembly with commercially available cochlear implant electrode arrays. The inner diameter of stylet tube 308 is smaller than 85 μm to ensure its stiffness and leave enough space for optical imaging probe 200. An optical fiber (e.g., first waveguide 208 and second waveguide 210) in sheath 300 has an outer diameter that is smaller than the inner diameter of sheath 300, specifically stylet tube 308. Optical probe head 212 has an outer diameter small than the inner diameter of the outer diameter of stylet tube 308. Stylet tube 308 has a length of longer than 20 mm. In some embodiments, endoscope system 100 functions in an assembly with an electrode array during the insertion of a cochlear implant. In these embodiments, the length of stylet tube 308 is important to ensure a deep insertion distance of the electrode array.

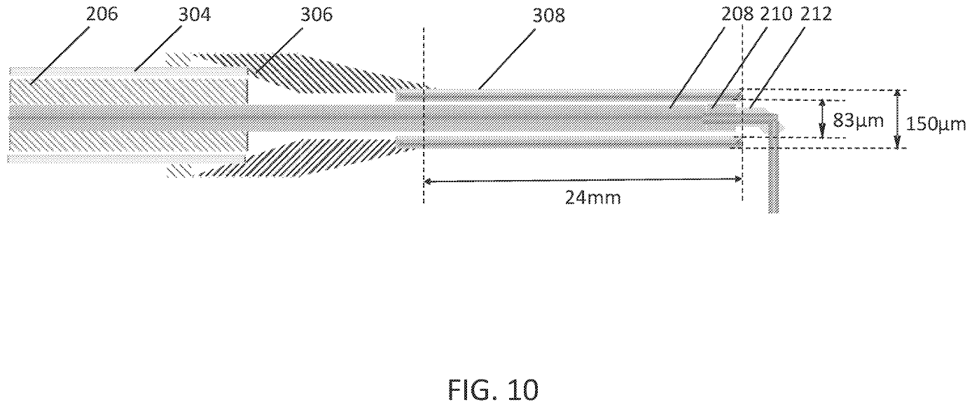

FIG. 10 shows a schematic of one embodiment of sheath 300. In this embodiment of sheath 300, stylet tube 308 has a length of 24 mm (±2 mm) exposed from tube adaptor 306 and has an outer diameter of 150 μm (±5 μm) and an inner diameter of 83 μm (±1 μm). First waveguide 208 has a length exceeding that of driveshaft assembly 206 that allows optical probe head 212 to advance beyond the distal end of stylet tube 308.

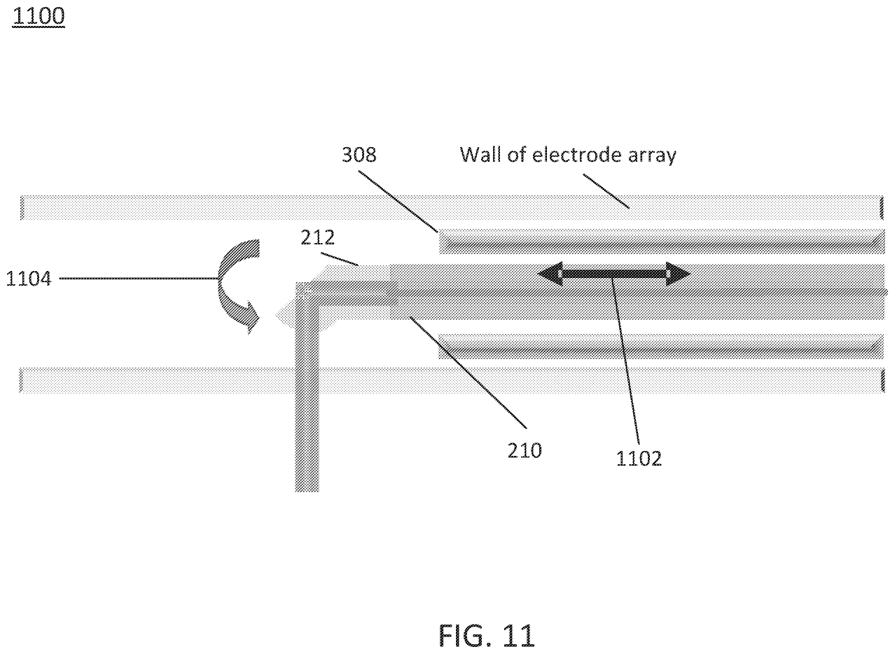

A key feature of endoscope device 100 is that the axial position of optical imaging probe 200, especially the position of optical probe head 212, relative to sheath 300, can be precisely controlled and adjusted by optical probe position adjustment module 204. When optical imaging probe 200 is rotating inside sheath 300, optical probe head 212 will not touch anything that may damage it (e.g., the wall of a cochlear implant electrode array). The smooth transitional surface of tube adaptor 306 ensures that optical imaging probe 200 can smoothly pass through the adaptor when being inserted and advanced (e.g., without getting caught on a rough or stepped edge). FIG. 11 shows a schematic of optical imaging probe 200 moving within sheath 300. Optical imaging probe 200 can move axially (axial movement 1102) and rotate about the center axis of optical imaging probe 200 (rotational movement 1104). Optical probe head 212 can reside within stylet tube 308 or be extended beyond the distal end of stylet tube 308.

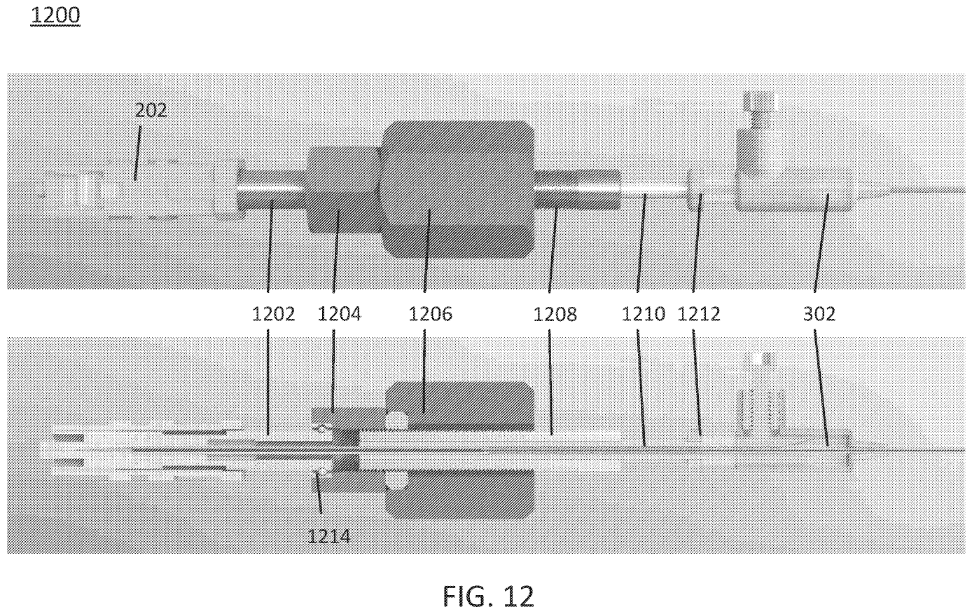

In one embodiment, optical probe adjustment module 204 further includes a mating sleeve 1202, a travel nut 1204, a locking nut 1206, a travel rode 1208 a sheath hypotube 1210, and a sheath adaptor 1212. FIG. 12 shows a schematic and cross-sectional view of an embodiment of optical position adjustment module 204, where travel nut 1204 may further include a bearing 1214 (FIG. 12, bottom panel) Mating sleeve 1202 connects to fiber connector 202 to the inner ring of bearing 1214 of travel nut 1204. Mating sleeve 1202 is also fixed to driveshaft assembly 206. Travel nut 1204 is fixed to the outer ring of bearing 1214 and couples to the fine thread on travel rode 1208. When travel nut 1204 is rotated along travel rod 1208, it will drive optical imaging probe 200 to traverse back and forth and can be used to advance optical probe head 212 out of or back within the distal end of stylet tube 308. Locking nut 1206 stops travel nut 1204 from rotating and pushing optical imaging probe 200 forward.

In one embodiment, driveshaft assembly 206 includes a stainless steel hypotube at its proximal end and a braided torque coil at its distal end, where the hypotube is fused to a torque coil. The hypotube at the proximal end is secured in fiber connector 202, mating sleeve 1102, and sheath hypotube 1110. The torque coil is secured within flexible sheath 304 and terminates before tube adaptor 306. Driveshaft assembly 206 secures first waveguide 208 inside and transmits torque at a 1:1 ratio from a rotary joint to optical probe head 212.

In some embodiments, optical probe position adjustment module 1200 is inserted into an electrode array. In some embodiments, optical imaging probe 200 is advanced by manually rotating travel nut 1104. In some embodiments, locking nut 1106 can be tightened towards travel nut 1104 to lock the position of optical imaging probe 200 inside sheath 300.

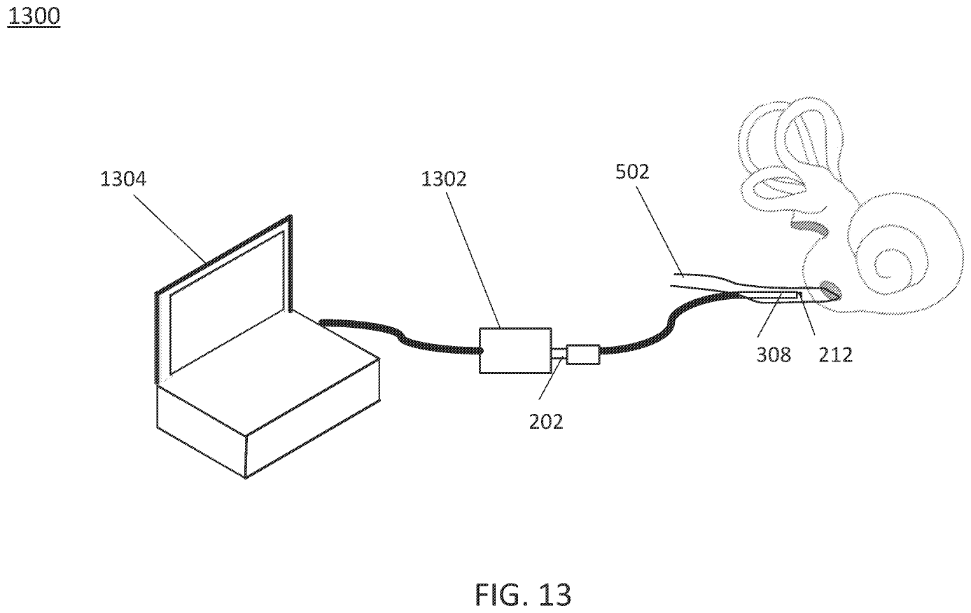

In some embodiments, endoscope device 100 or electrode array-endoscope device 500 works in conjunction with an OCT imaging console and a rotary joint. FIG. 13 shows a schematic of this configuration in accordance with one or more embodiments of the disclosure. In some embodiments, optical imaging probe 200 is connected to rotary joint 1302 via fiber connector 202. Rotary joint 1302 is further connected to OCT imaging console 1304. In some embodiments, an imaging laser from OCT console 1304 is delivered to endoscope device 100 through rotary joint 1302. Rotary joint 1302 drives optical imaging probe 100 to rotate and deliver a focused light beam on the circumference of the sample (e.g., the scala tympani of a human cochlea). The reflected optical information will be collected by OCT imaging console 1304 and OCT images will be processed and displayed in real-time.

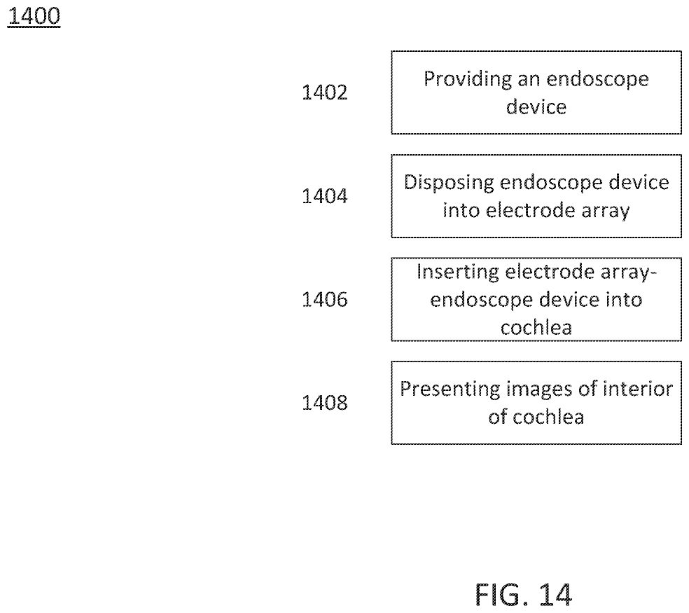

FIG. 14 shows an example 1400 of a process for micro-optical coherence tomography imaging of the cochlea in accordance with some embodiments of the disclosed subject matter. In some embodiments, the method comprises providing 1402 an endoscope device. In some embodiments, the endoscope device provided is consistent with one or more of the embodiments of endoscope device 100 described herein. In some embodiments, the endoscope device provided in 1402 comprises an optical imaging probe and a sheath. In some embodiments, the optical imaging probe can extend beyond the distal end of the sheath. In some embodiments, the method further comprises disposing 1404 the endoscope device into the channel of an electrode array. In some embodiments, the method further comprises inserting 1406 the electrode array-endoscope device from 1404 into the cochlea. In some embodiments, inserting 1406 further comprises emitting light into the proximal end of the optical imaging probe such that light is emitted toward the interior of the cochlea and focuses on tissue of the cochlea. In some embodiments, inserting 1406 further comprises rotating the optical probe to focus light on different locations of the cochlea. In some embodiments, inserting 1406 further comprises causing the optical imaging probe to receive reflected light from the cochlea. In some embodiments, inserting 1406 further comprises generating a two-dimensional image of a portion of the cochlea. In some embodiments, the method further comprises presenting 1408 images of the interior of a cochlea. In some embodiments, generated images are indicative of a distance between the outer surface of the optical imaging probe and a wall of the cochlea.

In some embodiments, endoscope device 100 works inside electrode arrays of cochlear implant systems 502 that utilize that “advance off-stylet” technique. “Advance off-stylet” is a cochlear implant electrode array insertion technique in which the electrode arrays are designed to have a channel in the center of the body and a solid metal stylet is pre-inserted into the channel to increase the assembly's rigidness for easier insertion and mitigate the chance of insertion trauma. The described endoscope device can replace the traditional solid stylet and act as a traditional stylet for insertion while simultaneously performing high-resolution OCT imaging through the electrode array body.

Before the imaging starts, endoscope device 200 will be inserted into the channel of a cochlear implant electrode array 502. This forms electrode array-endoscope device 500. During this procedure optical probe head 212 is secured and protected inside stylet tube 300 so it will not be damaged by the insertion. Once the cochlear implant array has been inserted, the optical imaging probe 200 will then be advanced within the sheath 300. The tip of optical imaging probe 200, specifically optical probe head 212, will stretch out from stylet tube 308, and rotate inside the electrode array's center channel without touching the inner wall of the electrode array due to stylet tube 308 maintaining a spacing between optical probe head 212 and the walls of electrode array 502 (see FIG. 11). The imaging will take place when the electrode array-endoscope device assembly 500 is being inserted together into the scala tympani, manually by an ENT surgeon or with a robotic insertion system. The optical imaging probe 200 will rotate inside the sheath 300, and the light beam will pass through the wall of electrode array and focus on the tissue of the cochlea. During manual and/or robotic insertion of a cochlear implant, the lumen and organ of Corti are visualized by embodiments of the disclosed procedures during and after insertion; visualization may be used to ensure that there is minimal (or no) damage to the basilar membrane during insertion; visualization may also be used to ensure proper placement of the cochlear implant; and/or the insertion process may be aided or automated by the addition of image guidance (e.g., machine vision), which may also speed up the insertion process and/or reduce the chance for injury during the process. Furthermore, the disclosed procedures may be used before, during, and/or after implantation to visualize the spiral ganglion neuron (SGN) to further optimize the performance of the implant.

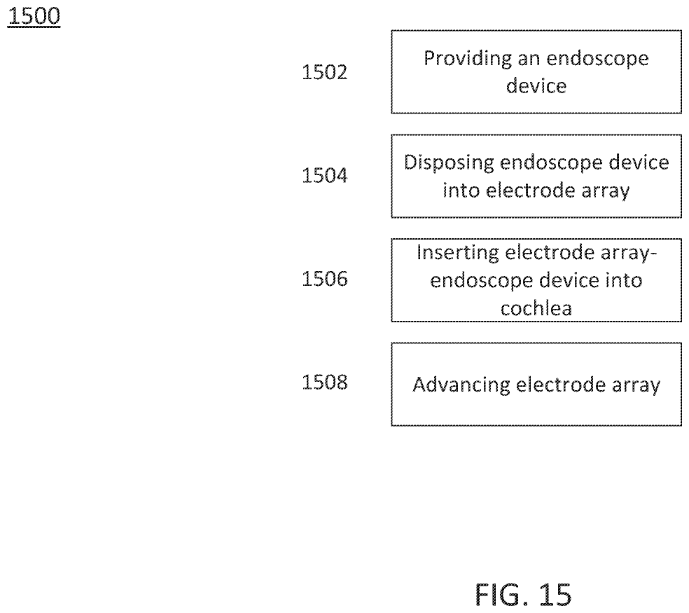

FIG. 15 shows an example 1500 of a process for imaging-guided cochlear implantation. In some embodiments, process 1500 comprises providing an endoscope device. In some embodiments, the endoscope device provided is consistent with one or more embodiments of endoscope device 100 described herein. In some embodiments, the endoscope device provided comprises an optical imaging probe and a sheath. In some embodiments, the optical imaging probe is capable of moving within the sheath and extending beyond the sheath. In some embodiments, process 1500 comprises disposing 1504 an endoscope device provided in 1502 into an electrode array. In some embodiments, an endoscope device provided in 1502 is configured such that the optical imaging probe is entirely within the sheath during disposing 1504. In some embodiments, the process further comprises inserting 1506 an electrode array-endoscope device from disposing 1502 into the cochlea. In some embodiments, the cochlea is a cochlea of a human. In some embodiments, inserting 1506 further comprises emitting light into the proximal end of the optical imaging probe such that the optical imaging probe emits light toward the interior of the cochlea and focusing on the tissue of the cochlea. In some embodiments, inserting 1506 further comprises rotating the optical imaging probe to focus on different locations of the tissue of the cochlea. In some embodiments, inserting further comprises causing the optical imaging probe to receive reflected light from the cochlea and transmit reflected light toward the optical imaging probe. In some embodiments, inserting further comprises generating a two-dimensional image of a portion of the cochlea. In some embodiments, inserting 1506 further comprises identifying an ideal insertion depth based on the generated two-dimensional images of the cochlea. In some embodiments, process 1500 further comprises advancing 1508 the electrode array from disposing 1504 off of the distal end of the endoscope device. In some embodiments, advancing comprises continuing to rotating the optical imaging probe and generating images in order to monitor the position of the electrode array and the cochlea.

When the electrode array-endoscope device assembly 500 reaches the desired insertion depth, the electrode array 502 will be advanced off of stylet tube 308. In some embodiments, stylet tube 308 continues rotating and imaging to monitor the position of electrode array 502 while electrode array 502 is being advanced off of stylet tube 308. In some embodiments, electrode array 502 is “pushed off from” stylet 308. In some embodiments, electrode array 502 is pushed off by surgeons using tweezers. After electrode array 502 is fully inserted, endoscope device 100 will be withdrawn from the electrode array and the cochlea. In some embodiments, endoscope device 100 will continue collecting images during withdrawal from the cochlea. This mitigates the risk of damaging the cochlea during withdrawal of the endoscope device.

The endoscope device enables real-time imaging, which can distinguish the outer wall of the electrode array and the surface of the scala tympani lumen to confirm that the electrode-endoscope assembly is being properly inserted. Endoscope device 100 enables the insertion of electrode array 502 to avoid touching and damaging the sensory tissue inside the cochlea. Real-time imaging can also provide cross-sectional images of the scala tympani, including morphology of the interior of the cochlea.

By pairing endoscope device 100 with current on-market cochlear implant electrode arrays, this OCT imaging can provide intraoperative guidance to the placement of electrode array. It can also obtain morphology information of the cochlea to diagnose the etiology of the patient's hearing loss, and/or make predictions of the prognosis of the patient.

It will be appreciated by those skilled in the art that while the disclosed subject matter has been described above in connection with particular embodiments and examples, the invention is not necessarily so limited, and that numerous other embodiments, examples, uses, modifications, and departures from the embodiments, examples, and uses are intended to be encompassed by the claims attached hereto. The entire disclosure of each patent and publication cited herein is hereby incorporated by reference, as if each such patent or publication were individually incorporated by reference herein.

Various features and advantages of the invention are set forth in the following claims.

What is claimed is:

1. An endoscope system, comprising:
an optical imaging probe comprising a proximal end and a distal end and comprising a fiber connector, an optical probe position module, an optical fiber, and an optical probe head,
the optical probe head being configured to deliver a focused light beam,
the optical probe position module controlling an axial position of the optical probe head; and
a sheath having a proximal end and a distal end, comprising a flexible sheath, a tube adaptor, and a stylet tube,
the flexible sheath, tube adaptor, and stylet tube being configured to form a continuous channel and surround at least a portion of the optical fiber.

2. The system of claim 1, wherein the optical fiber comprises a first waveguide and a second waveguide, and
wherein the first waveguide is located at the proximal end of the optical fiber and the second waveguide is located at the distal end of the optical fiber.

3. The system of claim 2, wherein the first waveguide is a single-mode fiber.

4. The system of claim 2, wherein the second waveguide is a multimode fiber.

5. The system of claim 1, wherein the optical probe head comprises a 3D printed monolithic structure including multiple optical surfaces, and
wherein the optical probe head is configured to generate multiple light propagation modes and a plurality of foci.

6. The system of claim 3, wherein the second waveguide comprises two segments of multimode fibers with different core diameters, configured such that the light beams expand before reaching the focusing element.

7. The system of claim 1, wherein the stylet tube comprises metal and is at least 20 millimeters long.

8. The system of claim 1, wherein the stylet tube is made of high-strength material and the stylet tube is transparent to optical coherence tomography light.

9. The system of claim 1, wherein a proximal end of the continuous channel is wider than a distal end of the continuous channel, and
wherein the tube adaptor comprises a reduction in diameter from a proximal end to a distal end thereof.

10. The system of claim 1, wherein the tube adaptor provides a change in outer diameter from the flexible sheath to the stylet tube, wherein an outer diameter of a distal end of the tube adaptor is smaller than an outer diameter of a proximal end of the tube adaptor.

11. The system of claim 1, wherein the optical probe head is disposed within the stylet tube and is configured to be advanced distally out of the distal end of the stylet tube.

12. The system of claim 1, wherein the fiber connector connects to a rotary joint.

13. The system of claim 1, wherein the optical probe position module houses a driveshaft assembly which is configured to rotate within the continuous channel and redirect the focused light beam.

14. The system of claim 1, the system further comprising a pre-curved electrode array or a non-pre-curved electrode array disposed over the sheath.

15. The system of claim 14, wherein the stylet tube supports the pre-curved electrode array and holds the pre-curved electrode array in a straight configuration.

16. A method of imaging a cochlea, comprising:
providing an endoscope device, comprising:
an optical imaging probe comprising a proximal end and a distal end and comprising a fiber connector, an optical probe position module, an optical fiber, and an optical probe head,
the optical probe head being configured to deliver a focused light beam,
the optical probe position module controlling an axial position of the optical probe head, and
a sheath having a proximal end and a distal end, comprising a flexible sheath, a tube adaptor, and a stylet tube,
the flexible sheath, tube adaptor, and stylet tube being configured to form a continuous channel and surround at least a portion of the optical fiber;
disposing the endoscope device into the channel of an electrode array;
inserting the endoscope device into the cochlea and advancing the endoscope device,
wherein inserting comprises:
emitting light into the proximal end of the optical imaging probe and causing the optical probe head to emit light toward the interior of the cochlea and focus on the tissues of the cochlea,
rotating the optical imaging probe to focus light on different locations of the cochlea, and
causing the optical imaging probe to receive reflected light from the cochlea and transmit the reflected light toward the proximal end of the optical imaging probe; and
generating a two-dimensional image of a portion of the cochlea surrounding the optical imaging probe based on the reflected light received from the optical imaging probe; and
causing the two-dimensional image of the portion of the cochlea surrounding the optical probe to be presented, wherein the two-dimensional image is indicative of a distance between an outer surface of the optical imaging probe and a wall of the cochlea.

17. The method of claim 16, wherein the generated two-dimensional images provide cross-sectional images of at least one of a scala tympani, a basilar membrane, or a basilar membrane structure.

18. The method of claim 16, further comprising:
obtaining, based on the generated two-dimensional images, morphology information of the cochlea to aid in diagnostic or prognostic predictions of hearing loss.

19. The method of claim 16, further comprising:
performing a pullback operation to extract the endoscope device from the cochlea, and
generating further two-dimensional images during the pullback operation.

20. The method of claim 16, wherein the optical probe head is retracted into the stylet tube, causing the light to perform a helical scan of the cochlea and generating three-dimensional images of the interior of the cochlea.

21. A method of imaging-guided cochlear implantation, comprising:
providing an endoscope device, comprising:
an optical imaging probe comprising a proximal end and a distal end and comprising a fiber connector, an optical probe position module, an optical fiber, and an optical probe head,
the optical probe head being configured to deliver a focused light beam,
the optical probe position module controlling an axial position of the optical probe head, and
a sheath having a proximal end and a distal end, comprising a flexible sheath, a tube adaptor, and a stylet tube,
the flexible sheath, tube adaptor, and stylet tube being configured to form a continuous channel and surround at least a portion of the optical fiber;
disposing the endoscope device into the channel of an electrode array;
inserting the electrode array-endoscope device into a cochlea and advancing the electrode array and the endoscope,
wherein inserting comprises:
emitting light into the proximal end of the optical imaging probe and causing the optical probe head to emit light toward the interior of the cochlea and focus on the tissue of the cochlea,
rotating the optical imaging probe to focus light on different locations of the tissue of cochlea,
causing the optical imaging probe to receive reflected light from the cochlea and transmit the reflected light toward the proximal end of the optical imaging probe, and
generating a two-dimensional image of a portion of the cochlea surrounding the optical imaging probe based on the reflected light received from the optical imaging probe;
identifying an insertion depth based on the generated two-dimensional images of the cochlea; and
advancing the electrode array off of the stylet tube, during which the optical imaging probe continues rotating and imaging to monitor the position of the electrode array and the cochlea.

22. The method of claim 21, wherein the inserting is performed by a human operator.

23. The method of claim 21, wherein the inserting is performed by a robotic insertion system.

24. The method of claim 21, further comprising:
distinguishing, based on the generated two-dimensional images, the outer wall of the electrode array and the surface of a scala tympani lumen to guide electrode array insertion.

25. The method of claim 21, further comprising:
providing, based on the generated two-dimensional images, cross-sectional images of a morphology of the cochlea.

26. The method of claim 21, further comprising:
performing a pullback operation to extract the endoscope device from the cochlea, and
generating further two-dimensional images during the pullback operation.

27. The method of claim 21, wherein the optical probe head is retracted into the stylet tube, causing the light to perform a helical scan of the cochlea and generating three-dimensional images of the interior of the cochlea.
